(19) United States
(12) Patent Application Publication
(10) Pub. No.: US 2006/0159698 A1
(43) Pub. Date: Jul. 20, 2006

(54) DRUG PRODUCT FOR INTESTINAL DISEASE

(76) Inventors: Yukie Murata, Kanagawa (JP); Junji Hamuro, Kanagawa (JP)

Correspondence Address: CERMAK & KENEALY LLP;ACS LLC, 515 EAST BRADDOCK ROAD, SUITE B, ALEXANDRIA, VA, 22314, US

(21) Appl. No.: 11/278,585

(22) Filed: Apr. 4, 2006

Related U.S. Application Data
(62) Division of application No. 10/720,169, filed on Nov. 25, 2003.
Application No. 10/720,169 is a continuation of application No. PCT/JP02/04980, filed on May 23, 2002.

(30) Foreign Application Priority Data
Jun. 1, 2001 (JP) 166982/2001

(51) Int. Cl. A61K 36/06; A61K 36/09; A61K 31/715

(52) U.S. Cl. 424/195.15; 514/54

(57) ABSTRACT

The present invention provides a drug product for the prevention, improvement in conditions relating to, and/or treatment of intestinal disease, comprising β(1-3) glucan derived from vegetable material and having a molecular weight of from about 5,000 to about 20,000. The present invention can be widely employed as a drug product for various intestinal diseases, particularly inflammatory bowel disease. The drug product of the present invention has almost no side effects, is highly effective when orally administered, and can be conveniently employed by numerous patients. Furthermore, the drug product of the present invention can be employed in the form of a health food product. The present invention also provides a method of preventing, improving conditions relating to, and/or treating intestinal disease.

This application claims the benefit as a divisional application under 35 U.S.C. §120 to application Ser. No. 10/720,169, filed Nov. 25, 2003, which is a continuation of PCT/JP02/04980, which was filed May 23, 2002, both of which are incorporated by reference in their entirety.

### BACKGROUND OF THE INVENTION

1. Technical Field

The present invention relates to a novel drug product specifically suited to the prevention, improvement in conditions relating to, and/or treatment of inflammatory bowel disease. It is highly safe, may be administered orally, and may thus be conveniently administered to a broad range of patients. The present invention further relates to a method of treating, improving conditions relating to, and/or preventing intestinal disease, and the use of the specific active components employed in this drug product in drug products for the prevention, improvement in conditions relating to, and/or treatment of intestinal disease.

2. Brief Description of the Related Art

Intestinal disease, particularly inflammatory bowel disease, is a disease with a growing number of victims for which the necessity of prevention and treatment has been increasing in recent years. The pervasiveness of intestinal bacteria and a compromised immune system are the assumed causes of this disease. However, the cause of onset is not clearly any one morbidity increasing mechanism. There is a large difference in morbidity between ulcerative colitis and Crohn's disease, both of which are typical inflammatory intestinal diseases. In the former, the contribution of stimulated humoral immunity exacerbation is high, while in the latter, stimulated cell-mediated immunity exacerbation is high. However, the details are unknown. Accordingly, the means of preventing this disease and improving morbidity are limited and unsatisfactory. Particularly from the perspective of prevention, nothing has been provided that satisfies the needs of the treatment domain.

Given the above-described situation, there is clearly a need in the art for the development of a drug product for intestinal disease, particularly a drug product, and food product, that can be widely employed for inflammatory bowel disease, that has almost no side effects, are effective when orally administered, and that can be conveniently employed by many patients. The problem solved by the present invention is the development of such a drug product and preventive food product.

### DISCLOSURE OF THE INVENTION

The present inventors conducted extensive research into solving the above-stated problem. They discovered that in model animal experiments in mice given drinking water containing β(1-3) glucan having a specific molecular weight derived from vegetable material, the above-described desirable pharmacological effects were achieved. They discovered that this specific glucan could be used as a drug product for preventing, improving conditions relating to, and treating intestinal disease. The present invention was devised on the basis of these discoveries.

It is an object of the present invention to provide a drug product for the prevention, improvement in conditions relating to, and/or treatment of intestinal disease, comprising β(1-3) glucan derived from vegetable material and having a molecular weight of from about 5,000 to about 20,000.

It is a further object of the present invention to provide a method of preventing, improving conditions relating to, and/or treating intestinal disease comprising administering to a body a drug product comprising β(1-3) glucan derived from vegetable material and having a molecular weight of from about 5,000 to about 20,000. The various above-described forms of the drug of the present invention may be employed in this administration.

The various above-described forms of the drug product of the present invention may be employed as the drug product used in the prevention, improvement in conditions relating to, and/or treatment of intestinal disease.

### BRIEF DESCRIPTION OF THE FIGURES

FIG. 1a shows the onset rate (%) (day 34 of switch to DSS 1% aqueous solution) of inflammatory intestinal disease (IBD) in Embodiment 1.

FIG. 1b shows the body weight reduction rate (%) (day 10 of switch to DSS 1% aqueous solution) in Embodiment 1.

In FIGS. 1a and 1b, the X-axis denotes, sequentially from the left, a control, Dry L60-min formic acid decomposed product, and Dry L30-min formic acid decomposed product.

FIG. 2 shows the survival rate (%) after the switch to DSS 2% aqueous solution in Embodiment 1. □: Control; ●: Dry L60-min formic acid decomposed product; A: Dry L30-min formic acid decomposed product.

FIG. 3 shows the change in body weight following the switch to DSS 2% aqueous solution in Embodiment 1. Y-axis: The ratio when the weight at the start of the switch to DSS 2% aqueous solution is taken as 1. □: Control; O: Dry L60-min formic acid decomposed product; ●: Dry L30-min formic acid decomposed product.

### DESCRIPTION OF THE PREFERRED EMBODIMENTS

The drug product of the present invention is not specifically limited to the prevention, improvement in conditions relating to, and treatment of intestinal disease, particularly inflammatory bowel disease. It is administered to mammals, usually humans.

The intestinal disease treated with the drug product of the present invention is not specifically limited; it is particularly effective against inflammatory bowel disease typified by Crohn's disease and ulcerative colitis.

The active component employed in the drug product of the present invention is β(1-3) glucan derived from vegetable material and having a molecular weight of from about 5,000 to about 20,000. The use of a component having a molecular weight denoted as an average molecular weight of from about 5,000-20,000 as the principal component is convenient. To prepare a glucan with a molecular weight falling within this range, a glucan of relatively high molecular weight is hydrolyzed to obtain a molecule of low molecular weight. For example, degradation with an enzyme such as β-(1-3) glucanase, chemically decomposed with formic acid or the like, or degradation via a physical method are all possible methods for preparing a glucan having a molecular weight falling within the desired range.

In the present invention, the term “β(1-3) glucan” includes all glucans having a β(1-3) bond, as well as glucans having a main chain in the form of a β(1-3) glusoside.

β(1-3) glucans obtained from mushrooms such as Matsutake [Tricholoma matsudake], Shiitake [Lentinus edodes], Bukuryo [Poria cocos], Kawaratake [Coriolus versicolor], Enokidake [Flammulina veltipes], Hiratake [Pleurotus ostreatus], Yamabushitake [Hericium erinaceum], and Agarikusuku [Agaricus blazei murrill] can be employed as the β(1-3) glucan derived from vegetable material. Such components can be readily prepared from mushrooms, for example, by obtaining an aqueous (hot water) extract, then precipitating with an alcohol (ethanol or the like) and, reducing the molecular weight thereof if necessary. Methods for obtaining a molecular weight falling within the above-stated range include hydrolysis by suitable methods (enzymatic decomposition, hydrolysis with an acid such as formic acid, and decomposition by physical methods) (see Sasaki et al., Gann, 67, 191-195, April, 1976.).

The drug product of the present invention is effective orally. The drug product is safe, and there is no effect with regard to obesity. Accordingly, the form of administration is not specifically limited, and includes ingestion via food products. Various forms of administration are possible, including oral administration and non-oral administration (intravenous administration and the like). The “body” to be administered to may be a mammal, and is preferably a human. Convenient, broad treatment of patients suffering from inflammatory bowel disease is possible. Due to safety and the suitability to oral administration, the drug product of the present invention can be employed in the form of health food products and therapeutic food products for prevention and improvement in such patients, as well as to patients having diseased intestines. It can also be widely administered in a preventative, therapeutic, and convenient fashion to patients who are at high risk for these diseases.

The present invention also permits mixing and combining with other drug product components (medically active substances). In such cases, so long as the active component of the present invention is present and the above-described targeted pharmacological activity is exhibited, the product is covered by the drug product of the present invention.

The incorporation of various substances that are pharmacologically acceptable in formulations (adjuvants and the like) is also possible. Formulation-use substances may be suitably selected based on the form of formulation. Examples are excipients, diluting agents, additives, anticaking agents, binders, coatings, lubricants, slipping agents, gloss-imparting agents, flavoring agents, sweetening agents, and solubilizing agents. Specific examples of formulation-use substances are magnesium carbonate, titanium dioxide, lactose, mannitol, other sugars, talc, milk protein, gelatin, starch, cellulose and its derivatives, animal and plant oils, polyethylene glycol, and solvents such as sterile water and monohydric and polyhydric alcohols such as glycerol.

The drug product of the present invention can be prepared in the above-described known forms as well as various medical drug formulations to be discovered in the future for, for example, oral administration, intraperitoneal administration, cutaneous administration, and inhalation. Known methods and methods developed in the future can be suitably employed to prepare various forms of medical drug formulations of the drug product of the present invention.

Examples of these types of medical drug formulations are suitable solid and liquid formulations, such as grains, powders, coated tablets, tablets, (micro)capsules, suppositories, syrups, juices, suspensions, emulsions, titrations, injection solutions, and formulations affording extended release of active substances.

It is necessary that the above-described component be incorporated in a quantity suitably large to exhibit its drug effect in the formulation of the present invention based on the above-listed formulations.

The dosage of the drug product of the present invention is suitably selected based on the severity and type of symptoms presented by the intestinal disease patient, the type of formulation, and the like. For example, in oral administration, based on the net weight of the active component, a daily dosage per patient of about 10 mg to 10 g is desirable, about 100 mg to 5 g is preferred, and about 500 to 2,000 mg is even more preferred. In severe cases, even larger doses are possible. In terms of administration frequency and intervals, one administration every few days or one administration a day are both possible. Usually, however, there are several administrations per day, perhaps divided into 2 to 4 administrations, preferably before meals. Further, when administered intravenously, a dosage of about one-tenth to one-twentieth that of the above-described oral administration dosage is sufficient.

The drug product of the present invention can be broadly administered preventively and amelioratively to combat intestinal disease, and to patients who already suffer from such diseases, as a health food product, therapeutic food product, or special health food product in patients' meals. When employed as a health food product or the like, the above-described orally administered formulation can be referred to and orally administrable components and additives required by health food products can be added in the preparation. In that case, the drug product of the present invention can be provided in the form of food products (including all items placed in the mouth and chewed, such as chewing gum, toothpaste), nutrition agents, infusion formulations, and the like. These are also encompassed by the drug product of the present invention. Therapeutic food products may be in any form, including solids and liquids.

The mechanism by which the low molecular weight β(1-3) glucan of the present invention acts on intestinal disease is presumed to be as follows. β(1-3) glucan is thought to mainly target macrophages among inflammatory cells. The well-known impregnation by macrophages of the mucous membranes of the intestines is observed in inflammatory bowel disease, and these macrophages are thought to intensify the immune response to bacteria in the intestines. β(1-3) glucan is thought to act on the macrophages, suppressing their immune response intensifying action.

As set forth above, a further mode of the present invention is a method of preventing, improving conditions relating to, and/or treating intestinal disease comprising administering to a body a drug product comprising β(1-3) glucan derived from vegetable material and having a molecular weight of from about 5,000 to about 20,000.

All of these modes of the present invention, including their particular administration, including ingestion, can be readily practiced based on the above-described description of the drug product of the present invention, the embodiments described further below, and, as necessary, with reference to the prior art.

### EXAMPLES

The present invention is described in detail below based on the examples and embodiments. However, the present invention is not limited to the examples and embodiments. The percentages employed in the examples and embodiments are weight percentages unless specifically stated otherwise.

Samples and Laboratory Animals

1) Dextran sodium sulfate; made by ICN Co., “Cat. 160110”

2) 13-Week C57/BL6 female mice (prepared by Japan Charles River)

Preparation of Mushroom Product Extracted with Hot Water and Decomposed with Formic Acid

1) 60-Minute Hydrolysis Product

To a two-liter, round-bottom three-necked flask equipped with reflux condenser, temperature gauge, and mechanical stirrer, 31.0 g of Dry L product obtained by hot water extraction of raw Shiitake followed by precipitation from alcohol, 620 mL of 80% formic acid was weighed out, and the mixture was placed in an oil bath with stirring preheated to 100° C. At 20 min, the temperature of the reaction solution reached 90° C., and stirring was continued for another 60 min. Subsequently, the reaction vessel was placed with being stirred in an ice water bath to stop the reaction. The reaction solution was cooled to room temperature, yielding a dark-brown gel-like substance. This was transferred to a two-liter eggplant-shaped flask. The solvent was evaporated under vacuum, yielding 50.4 g of a glue-like solid. To this were added two liters of pure water, the mixture was heated in a water bath to 60° C., the solid was dissolved to the extent possible, the mixture was left standing, and the clear supernatant was separated by decantation. To the undissolved portion in which was present a fibrous solid were added 800 mL of pure water and a household blender was employed to mix and pulverize for 60 min. To this was added the above-mentioned supernatant and the mixture was processed in an ultrasonic bath with ultrasound, yielding a uniform suspension. The suspension obtained was rapidly frozen in a dry-ice—alcohol bath and then freeze-dried, yielding 30.4 g of solid. A 0.22% (by weight) suspension of this solid had a pH of 4.51. The average molecular weight was 12,200.

The Dry L consisted of 1 kg of raw Shiitake (Lentinus edodes) dissolved in 5,000 mL of hot water and then precipitated (320 g) from 5,000 mL of ethanol.

2) 30-Min Hydrolysis Product

The same operation as above was conducted with the exception that the period of heating after reaching 90° C. was shortened to 30 min in the above-described method of preparing a 60 min hydrolysis product, yielding 30.8 g of solid. The 0.22% (by weight) suspension thereof had a pH of 4.86. The average molecular weight was 25,000.

Preparation of an Enzyme Decomposition Product of the Hot Water Extract of Mushroom

To a two-liter, round-bottom three-necked flask equipped with reflux condenser, temperature gauge, and mechanical stirrer were weighed out and charged 30.0 g of raw Shiitake hot-water extract. This was dissolved in 2 L of sterile water, commercial β-(1-3) glucanase was added, and the mixture was stirred at 30° C. and reacted. The reaction continued for 60 min. Subsequently, the reaction vessel was immersed in an ice water bath to stop the reaction. The reaction solution was transferred to a five-liter eggplant-shaped flask, the solvent was distilled off under vacuum, and distillation was stopped before solid precipitated. The clear supernatant was separated and processed in an ultrasonic bath with ultrasound, yielding a uniform, clear solution. The solution obtained was quickly frozen in a dry-ice—alcohol bath and then freeze dried, yielding 32.4 g of solid. A 0.22% (by weight) suspension of the solid had a pH of 6.52. The average molecular weight was 9,200.

Preparation of 0.02 Weight Percent Aqueous Solution of the Dry L Hydrolysis Product

2 g quantities of Dry L60-min formic acid hydrolysis product and Dry L30-min formic acid hydrolysis product were dissolved in one-liter quantities of sterile water (double distilled, followed by filtration with a millipore filter) and the pH values thereof were measured. The pH values were then adjusted to close to pH 7.0 with 1 normal sodium hydroxide aqueous solution to prepare Dry L 0.02 weight % aqueous solutions.

(Preparation of 0.1 Weight Percent Aqueous Solution of Dry L)

1 g of Dry L60-min formic acid decomposed product was dissolved in one liter of sterile water and the pH was measured. The pH was adjusted to close to 7.0 with a 1 normal sodium hydroxide aqueous solution to prepare a 0.1 weight % aqueous solution of Dry L.

Preparation of 1 Percent DSS Aqueous Solution

10 g of dextran sulfate sodium was dissolved in one liter of sterile water to prepare 1% DSS aqueous solution.

### EMBODIMENT 1

Test of Effect of Orally Ingested Dry L with Inflammatory Bowel Disease (DSS-Induced Inflammatory Bowel Disease) Model

Suppression of DSS-induced inflammatory bowel disease by the oral administration of Dry L decomposed product was confirmed. This is specifically described below.

The system where mice are made to drink an aqueous solution of dextran sulfate sodium (abbreviated to “DSS” hereinafter) is a known inflammatory bowel disease (abbreviated to “IBD” hereinafter) model. We employed this system to test whether the preadministration of an aqueous solution of Dry L60-min formic acid decomposed product would suppress or accelerate onset.

When IBD is induced, body weight decreased. Bloody feces, weight reduction, and the survival rate were employed as indicators of onset.

Female 13-week C57/BL6 mice were given drinking water in the form of an aqueous solution of Dry L60-min formic acid decomposed product and an aqueous solution of Dry L30-min formic acid decomposed product and a control group was given sterile water. On day 10 after the start of drinking water, the drinking water was switched to a DSS 1% aqueous solution. Body weight was then measured once a week and subsequent survival was tracked.

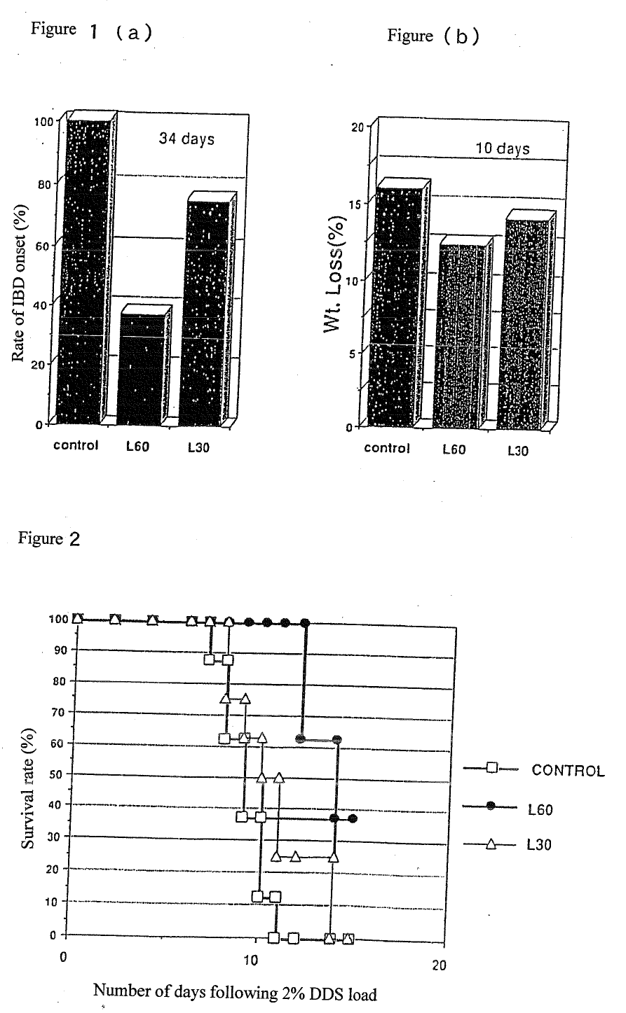

The diminution in body weight on day 10 following the switch to DSS 1% aqueous solution was 16% in the control group and 12.5% in the group given drinking water containing Dry L60-min formic acid decomposed product. A significant decrease in the group given drinking water containing L60-min formic acid decomposed product was confirmed (see FIG. 1b). The rate of onset (bloody feces) on day 34 following the switch to DSS 1% aqueous solution was 100% in the control group and 37% in the group given drinking water containing Dry L60-min formic acid decomposed product. Significant suppression of onset was confirmed in the group given drinking water containing Dry L60-min formic acid decomposed product. Further, in the group given drinking water containing Dry L30-min formic acid decomposed product, 75% onset rate % was confirmed as suppression of onset (see FIG. 1a).

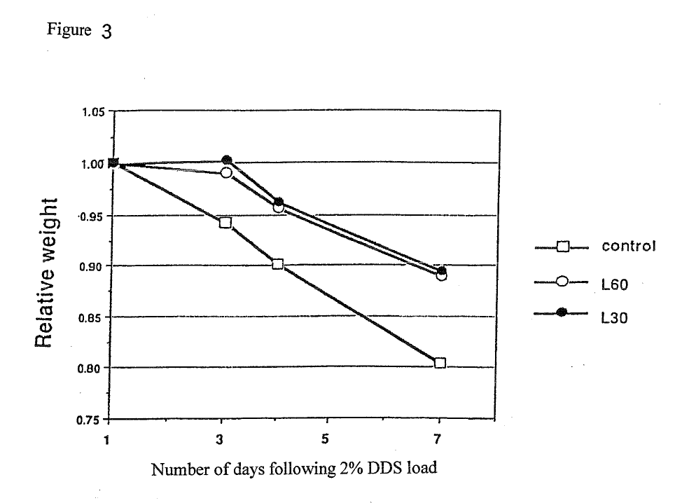

On day 34 following the switch to DSS 1% aqueous solution, a switch was made to DSS 2% aqueous solution. On day 11 after the switch to DSS 2% aqueous solution, the survival rate was 0% in the control group and 100% in the group given drinking water containing Dry L60-min formic acid decomposed product. Clear suppression of onset was confirmed in the group given drinking water containing L60-min formic acid decomposed product (see FIG. 2). On day 7 following the switch to DSS 2% aqueous solution, the change in body weight, relative to a body weight of 1 at the time of the initial switch, was 0.8 in the control group and 0.9 in the group given drinking water containing L60-min formic acid decomposed product. The reduction in body weight of the group given drinking water containing Dry L60-min formic acid decomposed product was thus less than that in the control group, confirming the suppression of onset of IBD (see FIG. 3).

As is clear from the above results, the ingestion of drinking water containing Dry L (60-min formic acid decomposed product) significantly suppresses inflammation in the IBD model.

### EMBODIMENT 2

Test of Effect of Orally Ingested Enzymatic Decomposition Product with Inflammatory Intestinal Disease (DSS-Induced Inflammatory Bowel Disease) Model

Suppression of DSS-induced inflammatory bowel disease by the oral ingestion of enzymatic decomposition product of Shiitake hot-water extract was confirmed. This is specifically described below.

The system where mice are made to drink an aqueous solution of dextran sulfate sodium (abbreviated to “DSS” hereinafter) is a known inflammatory bowel disease (abbreviated to “IBD” hereinafter) model. We employed this system to test whether the preadministration of an aqueous solution of Shiitake hot water extract enzymatic decomposition product would suppress or accelerate onset.

Female 13-week C57/BL6 mice were given drinking water containing Shiitake hot water extract enzymatic decomposition product and a control group was given sterile water. On day 10 after the ingestion of drinking water, the drinking water was switched to a DSS 1% aqueous solution. Body weight was then measured once a week and the subsequent survival rate was tracked.

The diminution in body weight on day 10 following the switch to DSS 1% aqueous solution was 19% in the control group and 9.5% in the group given drinking water containing Shiitake hot water extract enzymatic decomposition product. A significant decrease in the group given drinking water containing Shiitake hot water extract enzymatic decomposition product was confirmed. The rate of onset (bloody feces) on day 30 following the switch to DSS 1% aqueous solution was 100% in the control group and 17% in the group given drinking water containing Shiitake hot water extract enzymatic decomposition product. Significant suppression of onset was confirmed in the group given drinking water containing Shiitake hot water extract enzymatic decomposition product.

The above results show that the ingestion of drinking water containing Shiitake hot water extract enzymatic decomposed product significantly suppressed inflammatory bowel disease relative to the control group.

The present invention provides a drug product suited to administration for the prevention, improvement in conditions relating to, and treatment of bowel disease, that can be employed as a drug product for intestinal disease, particularly inflammatory bowel disease, has almost no side effects, is highly effective when orally administered, and can be conveniently employed by large numbers of patients. It is highly safe and can be provided in the form of a health food. The present invention further provides a method of preventing, improving conditions relating to, and/or treating bowel disease, and the use of the above-described specific glucan as an active component in such drug products.

Accordingly, the present invention can be widely implemented in the fields of medical drugs, food products, medical treatment, feeds, and veterinary drugs, and is thus extremely useful from an industrial perspective.

While the invention has been described in detail with reference to preferred embodiments thereof, it will be apparent to one skilled in the art that various changes can be made, and equivalents employed, without departing from the scope of the invention. Each of the aforementioned documents, as well as the foreign priority document JP2001-166982 are hereby incorporated by reference.

1. A method of preventing, improving conditions relating to, and/or treating bowel disease comprising administering to a body a drug product comprising β(1-3) glucan derived from vegetable material and having a molecular weight of from 5,000 to 20,000.

2. The method of claim 1, wherein said β(1-3) is derived from a Shiitake mushroom.

3. The method of claim 1, wherein said drug product is suitable for oral administration.

4. A method of preventing, improving conditions relating to, and/or treating bowel disease comprising administering to a body a drug product comprising β(1-3) glucan derived from a Shiitake mushroom and having a molecular weight of from 5,000 to 20,000.
